(19) United States
(12) Patent Application Publication
(10) Pub. No.: US 2007/0135760 A1
(43) Pub. Date: Jun. 14, 2007

(54) Occlusion clearance in microsurgical system

(76) Inventor: David L. Williams, Newport Beach, CA (US)

Correspondence Address: ALCON, IP LEGAL, TB4-8, 6201 SOUTH FREEWAY, FORT WORTH, TX, 76134, US

(21) Appl. No.: 11/300,044

(22) Filed: Dec. 14, 2005

(51) Int. Cl. A61M 31/00

(52) U.S. Cl. 604/65

(57) ABSTRACT

A microsurgical system capable of clearing an occlusion via hydraulic resonance in its aspiration circuit.

### FIELD OF THE INVENTION

The present invention generally pertains to clearing occlusions in microsurgical systems and more particularly to clearing occlusions in ophthalmic microsurgical systems.

### DESCRIPTION OF THE RELATED ART

During small incision surgery, and particularly during ophthalmic surgery, small probes are inserted into the operative site to cut, remove, or otherwise manipulate tissue. During these surgical procedures, fluid is typically infused into the eye, and the infusion fluid and tissue are aspirated from the surgical site. These probes have small orifices that are easily clogged with tissue. Such clogging is typically referred to as “occlusion”, “tip occlusion”, or “port occlusion”. The process of clearing such occlusions is typically referred to as “reflux”.

A traditional method of reflux is to create a backpressure pulse of fluid that travels through the aspiration circuit to the tip or port of the probe to clear the incarcerated tissue. Because a single pulse of fluid often does not clear the occlusion, a surgeon must typically utilize a series of pulses until he or she visually observes the tip or port of the probe to be clear through the operating microscope. These traditional methods often create a large, steady-state pressurization of the aspiration circuit, which results in an undesirable infusion of pressurized fluid into the eye once the occlusion is cleared.

Therefore, a need continues to exist for an improved method of clearing an occlusion in a microsurgical system.

### SUMMARY OF THE INVENTION

The present invention provides improved apparatus and methods for clearing an occlusion in a microsurgical system.

In one aspect of the present invention, a microsurgical system is provided that includes an aspiration circuit containing a fluid disposed therein and a surgical device having a port for aspirating tissue fluidly coupled to the aspiration circuit. A series of hydraulic pressure pulses is created in the fluid at substantially the resonance frequency of the fluid in the aspiration circuit.

In another aspect of the present invention, a microsurgical system is provided that includes an aspiration circuit containing a fluid disposed therein and a surgical device having a port for aspirating tissue fluidly coupled to the aspiration circuit. A series of hydraulic vacuum pulses are created in the fluid at substantially the resonance frequency of the fluid in the aspiration circuit.

### BRIEF DESCRIPTION OF THE DRAWINGS

For a more complete understanding of the present invention, and for further objects and advantages thereof, reference is made to the following description taken in conjunction with the accompanying drawing, in which

FIG. 1 is a schematic diagram illustrating an aspiration circuit of a microsurgical system.

### DETAILED DESCRIPTION OF THE PREFERRED EMBODIMENTS

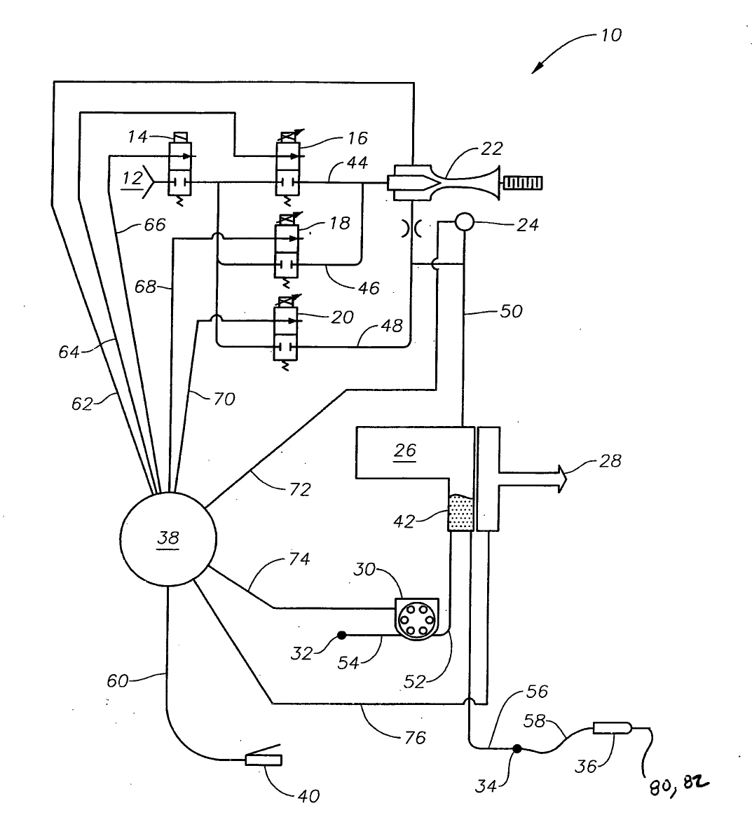

The preferred embodiment of the present invention and its advantages is best understood by referring to FIG. 1 of the drawings. Microsurgical system 10 preferably includes a pressurized gas source 12, an isolation valve 14, a vacuum proportional valve 16, an optional second vacuum proportional valve 18, a pressure proportional valve 20, a vacuum generator 22, a pressure transducer 24, an aspiration chamber 26, a fluid level sensor 28, a pump 30, a collection bag 32, an aspiration port 34, a surgical device 36, a computer or microprocessor 38, and a proportional control device 40. The various components of system 10 are fluidly coupled via fluid lines 44, 46, 48, 50, 52, 54, 56, and 58. The various components of system 10 are electrically coupled via interfaces 60, 62, 64, 66, 68, 70, 72, 74, and 76. Valve 14 is preferably an “on/off” solenoid valve. Valves 16-20 are preferably proportional solenoid valves. Vacuum generator 22 may be any suitable device for generating vacuum but is preferably a vacuum chip or a venturi chip that generates vacuum when isolation valve 14 and vacuum proportional valves 16 and/or 18 are open and gas from pressurized gas source 12 is passed through vacuum generator 22. Pressure transducer 24 may be any suitable device for directly or indirectly measuring pressure and vacuum. Fluid level sensor 28 may be any suitable device for measuring the level of a fluid 42 within aspiration chamber 26 but is preferably capable of measuring fluid levels in a continuous manner. Pump 30 may be any suitable device for generating vacuum but is preferably a peristaltic pump, a scroll pump, or a vane pump. Microprocessor 38 is capable of implementing feedback control, and preferably PID control. Proportional controller 40 may be any suitable device for proportionally controlling system 10 and/or surgical device 36 but is preferably a foot controller. Surgical device 36 may be any surgical device that aspirates tissue but is preferably an ophthalmic surgical device such as a phacoemulsification probe, a vitrectomy probe, or an aspiration probe. Surgical device 36 has a tip 80 with a port 82 that is fluidly coupled to fluid line 58.

The aspiration circuit of FIG. 1 enables an improved method of clearing an occlusion in a microsurgical system according to the present invention. Microprocessor 38 is capable of detecting an occlusion of port 82, such as, by way of example, by pressure transducer 24 measuring a predefined increase in the pressure of the aspiration circuit and sending a signal to microprocessor 38 via interface 72. When microprocessor 38 detects an occlusion of port 82, it creates a hydraulic resonance in the aspiration circuit having a frequency substantially matching the natural resonance frequency of the fluid in the aspiration circuit. This hydraulic resonance exerts a cyclic force on the occlusion that clears port 82 of the occlusion. Significantly, port 82 is cleared of the occlusion without the necessity of creating a large, steady-state pressurization of the aspiration circuit, which often results in an undesirable infusion of pressurized fluid into the eye once the occlusion is cleared.

When microprocessor 38 detects an occlusion of port 32, it can create such hydraulic resonance using a series of net positive pressure pulses or a series of net negative pressure pulses (vacuum pulses). For net positive pressure pulses, microprocessor 38 preferably activates and opens valves 14 and 20. Microprocessor then cycles valve 20 from an open position to a closed position at a frequency so as to create a series of pneumatic pressure pulses within fluid lines 48 and 50 that in turn create a series of hydraulic pressure pulses within fluid 42 within aspiration chamber 42, fluid lines 56 and 58, and probe 36 at or near the resonance frequency of the fluid 42 in the aspiration circuit. The amplitude of each pressure pulse can be adjusted via the degree to which microprocessor 38 opens proportional valve 20 in each cycle. When the resulting hydraulic resonance clears the occlusion from port 82 (as determined, for example, via a predefined pressure decrease in the aspiration circuit measured by pressure transducer 24), microprocessor halts the hydraulic resonance by closing valves 14 and 20.

For vacuum pulses, microprocessor 38 preferably activates and opens valves 14 and 16. Microprocessor then cycles valve 16 from an open position to a closed position at a frequency so as to create a series of pneumatic vacuum pulses within fluid lines 44 and 50 that in turn create a series of hydraulic vacuum pulses within fluid 42 within aspiration chamber 42, fluid lines 56 and 58, and probe 36 at or near the resonance frequency of the fluid 42 in the aspiration circuit. The amplitude of each vacuum pulse can be adjusted via the degree to which microprocessor 38 opens proportional valve 16 in each cycle. For higher levels of vacuum, microprocessor can optionally utilize valve 18 in addition to valve 16 to augment hydraulic resonance in a manner similar to valve 16. When the resulting hydraulic resonance clears the occlusion from port 82 (as determined, for example, via a predefined pressure decrease in the aspiration circuit measured by pressure transducer 24), microprocessor halts the hydraulic resonance by closing valves 14, 16, and 18.

From the above, it may be appreciated that the present invention provides an improved method of clearing an occlusion in a microsurgical system. In contrast to traditional methods, an injection of high pressure fluid into the eye upon the clearing of the occlusion is minimized or eliminated. Therefore, clearing an occlusion is safer for the patient.

The present invention is illustrated herein by example, and various modifications may be made by a person of ordinary skill in the art. For example, while the present invention is described above relative to clearing an occlusion in an ophthalmic microsurgical system, it is also applicable to other microsurgical systems. As another example, valves 16, 18, and 20 may be “on/off” solenoid valves instead of proportional solenoid valves. As a further example, valves 16 and 18 may each be fluidly coupled to a vacuum pump on a first side and to aspiration chamber 26 on a second side, and pressurized gas source 12 may be fluidly coupled only to valve 20. Vacuum generator 22 is eliminated. In this alternative, the vacuum pump would create the vacuum necessary for creating the series of pneumatic vacuum pulses. An optional vacuum receiver may be fluidly coupled to the vacuum pump for greater vacuum energy. As a further example, the hydraulic resonance necessary to clear an occlusion of port 82 may be created using a combination of net positive pressure pulses and net negative pressure (vacuum) pulses. In this alternative, the hydraulic pressure pulses and the hydraulic vacuum pulses may be appropriately timed to create an alternating “push-pull” effect to clear an occlusion of port 82.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. Apparatus for clearing an occlusion in a microsurgical system, comprising:
a pressurized gas source;
a surgical device having a port for aspirating tissue;
an aspiration chamber fluidly coupled between said pressurized gas source and said surgical device, said aspiration chamber and said surgical device containing a fluid disposed therein;
a valve fluidly coupled between said pressurized gas source and said aspiration chamber;
a pressure transducer fluidly coupled between said pressurized gas source and said aspiration chamber; and
a computer electrically coupled to said pressurized gas source, said valve, and said pressure transducer;
whereby upon a detection of an occlusion of said port by said pressure transducer, said computer cycles said valve from an open position to a closed position at a frequency so as to create a series of pneumatic pressure pulses that creates a series of hydraulic pressure pulses on said fluid disposed in said aspiration chamber and said surgical device at substantially a resonance frequency of said fluid.

2. The apparatus of claim 1 wherein said series of hydraulic pressure pulses clears said occlusion.

3. The apparatus of claim 1 wherein said valve is a proportional valve, and said computer is capable of adjusting an amplitude of said pneumatic pressure pulses via said proportional valve.

4. The apparatus of claim 1 wherein said microsurgical system is an ophthalmic microsurgical system, and said surgical device is an ophthalmic surgical device.

5. A method of clearing an occlusion in a microsurgical system, comprising the steps of:
providing a microsurgical system comprising an aspiration circuit containing a fluid disposed therein and a surgical device having a port for aspirating tissue fluidly coupled to said aspiration circuit; and
creating a series of hydraulic pressure pulses in said fluid at substantially a resonance frequency of said fluid in said aspiration circuit.

6. The method of claim 5 further comprising the step of clearing an occlusion of said port using said series of hydraulic pressure pulses.

7. The method of claim 5 wherein said microsurgical system is an ophthalmic microsurgical system, and said surgical device is an ophthalmic surgical device.

8. Apparatus for clearing an occlusion in a microsurgical system, comprising:
a pressurized gas source;
a vacuum generator fluidly coupled to said pressurized gas source;
a surgical device having a port for aspirating tissue;
an aspiration chamber fluidly coupled between said vacuum generator and said surgical device, said aspiration chamber and said surgical device containing a fluid disposed therein;
a valve fluidly coupled between said pressurized gas source and said vacuum generator;
a pressure transducer fluidly coupled between said pressurized gas source and said aspiration chamber; and
a computer electrically coupled to said pressurized gas source, said valve, and said pressure transducer;
whereby upon a detection of an occlusion of said port by said pressure transducer, said computer cycles said valve from an open position to a closed position at a frequency so as to create a series of pneumatic vacuum pulses that creates a series of hydraulic vacuum pulses on said fluid disposed in said aspiration chamber and said surgical device at substantially a resonance frequency of said fluid.

9. The apparatus of claim 8 wherein said series of hydraulic vacuum pulses clears said occlusion.

10. The apparatus of claim 8 wherein said valve is a proportional valve, and said computer is capable of adjusting an amplitude of said pneumatic vacuum pulses via said proportional valve.

11. The apparatus of claim 8 wherein said microsurgical system is an ophthalmic microsurgical system, and said surgical device is an ophthalmic surgical device.

12. A method of clearing an occlusion in a microsurgical system, comprising the steps of:
providing a microsurgical system comprising an aspiration circuit containing a fluid disposed therein and a surgical device having a port for aspirating tissue fluidly coupled to said aspiration circuit; and
creating a series of hydraulic vacuum pulses in said fluid at substantially a resonance frequency of said fluid in said aspiration circuit.

13. The method of claim 12 further comprising the step of clearing an occlusion of said port using said series of hydraulic vacuum pulses.

14. The method of claim 12 wherein said microsurgical system is an ophthalmic microsurgical system, and said surgical device is an ophthalmic surgical device.

15. The method of claim 12 further comprising the step of creating a series of hydraulic pressure pulses in said fluid at substantially a resonance frequency of said fluid in said aspiration circuit.

16. The method of claim 15 wherein said step of creating a series of hydraulic vacuum pulses and said step of creating a series of hydraulic pressure pulses are performed in an alternating manner.
